(19) United States
(12) Patent Application Publication
(10) Pub. No.: US 2007/0244190 A1
(43) Pub. Date: Oct. 18, 2007

(54) Neobritannilactone B and acetyl neobritannilactone B

(76) Inventors: Chi-Tang Ho, East Brunswick, NJ (US); Naisheng Bai, Highland Park, NJ (US); Min-Hsiung Pan, Kaohsiung City (TW)

Correspondence Address: JONES DAY, 222 EAST 41ST ST, NEW YORK, NY, 10017, US

(21) Appl. No.: 11/724,844

(22) Filed: Mar. 16, 2007

Related U.S. Application Data
(60) Provisional application No. 60/783,423, filed on Mar. 17, 2006.

(51) Int. Cl. A61K 31/365

(52) U.S. Cl. 514/469

(57) ABSTRACT

Provided herein are methods of inducing apoptosis in cancer cells using neobritannilactone B (NAB) or acety neobritannilactone B (ANAB). Also provided are pharmaceutical compositions and methods for using NAB or ANAB to prevent or treat cancer in a mammal. Exemplary cancers include, for example, colon cancer, leukemia, and gastric cancer.

### 1. CROSS-REFERENCE

This application claims the benefit of U.S. Provisional Application No. 60/783,423, filed Mar. 17, 2006, the content of which is incorporated herein by reference in its entirety.

### 2. FIELD OF THE INVENTION

Provided herein are pharmaceutical compositions and methods using neobritannilactone B (NAB) or acetyl neobritannilactone B (ANAB) to prevent or treat cancer in a mammal. Also provided are methods of inducing apoptosis in cancer cells using NAB or ANAB.

### 3. BACKGROUND OF THE INVENTION

Natural plant extracts have been shown to have activity as chemoprotective agents. An example, taxol, isolated from the Pacific yew tree Taxus brevifolia, acts by inducing Bcl-2 phosphorylation in cancer cells which leads to programmed cell death. See, e.g., Haldar et al. (1996) Cancer Res. 56, 1253-1255. Inula britannica, a wild plant found in Eastern Asia, including China, Korea and Japan is of interest since extracts of I. britannica are reported to have anti-tumor activities. See, e.g., Jiangsu New Medical College. Dictionary of Traditional Chinese Materia Medica, Vol. 2. (Shanghai People's Press: Shanghai, PRC, 1977), pp. 2216-2219. Various sesquiterpene lactones isolated from I. britannica have been shown to be cytotoxic. See Park and Kim (1998) Planta Med. 64, 752-754; Rafi et al. (2005) Anticancer Res. 25, 313-318; and U.S. Pat. No. 6,627,623. However, extracts of I. britannica are believed to contain a number of compounds with as yet undetermined activities.

Additional candidate therapies effective for the prevention and treatment of cancer are sought.

### 4. SUMMARY OF THE INVENTION

In one aspect, provided herein are compositions useful for their cytotoxic effects on cancer cells. In certain embodiments, the compositions provided are dietary supplements or nutraceutical compositions. In some embodiments, the compositions provided are pharmaceutical compositions.

In certain embodiments, pharmaceutical compositions provided herein comprise neobritannilactone B (NAB) and a pharmaceutically acceptable carrier, diluent or excipient.

In other embodiments, pharmaceutical compositions provided herein comprise acetyl neobritannilactone B (ANAB) and a pharmaceutically acceptable carrier, diluent or excipient.

In certain embodiments, the present invention provides compositions comprising purified NAB or purified ANAB, an isolated NAB or isolated ANAB or a purified and isolated NAB or purified and isolated ANAB.

In certain embodiments, compositions of the invention include plant extract comprising NAB and/or ANAB, wherein the concentration of NAB and/or concentration of ANAB relative to one or more components in the plant extract are greater than that found in the natural source from which the plant extract was derived.

In another aspect, provided herein are methods of inducing apoptosis in a cancer cell comprising contacting the cancer cell with an amount of neobritannilactone B (NAB) or acetyl neobritannilactone B (ANAB) sufficient to induce apoptosis.

In certain embodiments, the cancer cell is a colon cancer cell, a leukemia cell or a gastric cancer cell. In some embodiments, the cancer cell is contacted in vitro. In other embodiments, the cancer cell is contacted in vivo.

In one aspect, provided herein are methods for inhibiting the proliferation of a cancer cell in a mammal in need thereof, comprising administering to the mammal an amount of NAB or ANAB effective to inhibit proliferation of the cancer cell.

In one aspect, provided herein are methods for preventing, managing or treating a cancer in a mammal in need thereof, comprising administering to the mammal an amount of a NAB or ANAB effective to prevent or treat the cancer.

### 5. DESCRIPTION OF THE FIGURES

FIG. 1 provides models depicting the nuclear Overhauser enhancement spectroscopy (NOESY) correlations used to identify neobritannilactone B (NAB) and acetyl neobritannilactone B (ANAB) isolated from I. britannica extract (A) and structures of NAB and ANAB (B).

FIG. 2 provides results of the viability of AGS cells treated with NAB and ANAB as demonstrated by trypan blue dye exclusion of cells treated with the indicated concentrations of NAB or ANAB for 24 hours.

FIG. 3 provides results from a determination of sub-GI cells in NAB- or ANAB-treated AGS cells utilizing flow cytometry.

FIG. 4 provides results showing the induction of DNA fragmentation in AGS cells treated with 0-25mM of NAB and ANAB for 24 h. Internucleosomal DNA fragmentation was analyzed by agarose gel electrophoresis.

FIG. 5 provides light microscopic images showing the morphological changes of AGS cells, including the induction of apoptotic bodies from AGS cells, induced by NAB and ANAB.

FIG. 6 provides western blots using anti-poly-(ADP-ribose) polymerase (PARP) antibodies demonstrating a dose-dependent cleavage of PARP induced by NAB and ANAB (A), and a time-dependent cleavage of PARP induced by ANAB (B).

FIG. 7 provides western blots demonstrating the effects of various concentrations of NAB or ANAB on Bcl-xL expression in AGS cells (A), and effects of treating cells with 15 μM ANAB for various times (B).

### 6. TERMINOLOGY

Abbreviations used herein include: ANAB, acetyl neobritannilactone B; Bcl-2, B-cell leukemia/lymphoma 2; Bcl-X_{L}, B-cell leukemia/lymphoma extra long; COSY, correlation spectroscopy; DEPT, distortionless enhancement by polarization transfer; HMQC, heteronuclear multiple-quantum ccoherence; HMBC, heteronuclear multiple quantum coherence; HREIMS, high resolution electron ionization mass spectroscopy; HRFABMS, high resolution fast atom bombardment mass spectroscopy; IR, infrared spectroscopy; kDa, kilodalton; NAB, neobritannilactone B; NOESY, nuclear Overhauser enhancement spectroscopy; NMR, nuclear magnetic resonance spectroscopy; PARP, poly-(ADP-ribose) polymerase;

The term “about” as used herein refers to a value that is no more than 10% above or below the value being modified by the term. For example, the term “about 5%” means a range of from 4.5% to 5.5%.

As used herein, the term “composition” is meant to encompass dietary supplements, food additives, nutraceuticals, pharmaceutical compositions and physiologically acceptable compositions. It will be understood that where a component, for example, NAB or ANAB, in a “composition” also occurs in a natural source the term “composition” does not include the natural source of the component, but can, in certain embodiments, encompass a physically or chemically modified or processed form of the natural source, such as an extract of the natural source.

The term “effective amount” as used herein refers to the amount of NAB or ANAB that is sufficient to produce a desirable or beneficial effect when contacted, for example, to a cancer cell, or, as another example, when administered to a subject having a cancer. In certain embodiments, an “effective amount” is the amount of a NAB or ANAB sufficient to reduce or ameliorate the severity or duration of a cancer or a symptom thereof, prevent the advancement of a cancer, cause regression of a cancer, prevent the recurrence, development, or onset of a cancer or symptom associated with a cancer.

As used herein, the term “isolated” in the context of a compound or composition that can be obtained from a natural source, e.g., plants, refers to a compound or composition that is separated from one or more components from its natural source, preferably, a compound or composition that is substantially free of natural source cellular material, e.g., plant cellular material, or contaminating materials from the natural source, e.g., cell or tissue source, from which it is obtained. The language “substantially free of natural source cellular material” or substantially free of plant cellular material” includes preparations of a compound that has been separated from cellular components of the cells from which it is isolated. Thus, an “isolated” compound or composition is in a form such that its concentration or purity is greater than that in its natural source. For example, in certain embodiments, an “isolated” compound or composition can be obtained by purifying or partially purifying the compound or composition from a natural source. In some embodiments, an “isolated” compound or composition is obtained in vitro in a synthetic, biosynthetic or semisynthetic organic chemical reaction mixture.

As used herein, the terms “manage,” “managing,” and “management” refer to the beneficial effects that a subject with cancer derives from being administered with NAB or ANAB, if not resulting in a cure of the disease. In certain embodiments, a subject is administered NAB or ANAB to “manage” a disease so as to prevent the progression or worsening of the disease.

As used herein, the terms “prevent,” “preventing” and “prevention” refer to the prevention of the recurrence, onset, or development of a cancer or a symptom thereof in a subject resulting from the administration of NAB or ANAB the subject.

As used herein, the term “therapeutically effective amount” refers to that amount of NAB or ANAB sufficient to result in the amelioration of one or more symptoms of a cancer, prevent advancement of a cancer, cause regression of a cancer, or to enhance or improve the therapeutic effect(s) of another therapy. In a specific embodiment, an effective amount refers to the amount of NAB or ANAB that inhibits or reduces the proliferation of cancerous cells, inhibits or reduces the spread of tumor cells (metastasis), inhibits or reduces the onset, development or progression of cancer or a symptom thereof, induces apoptosis in cancerous cells or reduces the size of a tumor. Preferably, a therapeutically effective amount of NAB or ANAB reduces the proliferation of cancerous cells or the size of a tumor by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, relative to a control or placebo such as phosphate buffered saline (“PBS”).

As used herein, the terms “treat”, “treatment” and “treating” refer to the reduction or amelioration of the progression, severity and/or duration of a cancer, or the amelioration of one or more symptoms thereof resulting from the administration of NAB or ANAB. In specific embodiments, such terms refer to the inhibition or reduction in the proliferation of cancerous cells, the inhibition or reduction in the spread of tumor cells (metastasis), the inhibition or reduction in the onset, development or progression of cancer or a symptom thereof, the inducement of apoptosis in cancerous cells, the reduction in the size of a tumor, or the improvement in a patient's ECOG or Kamofsky score. In yet other embodiments, such terms refer to a reduction a human's PASI score or an improvement in a human's global assessment score.

### 7. DETAILED DESCRIPTION

### 7.1. Compositions Comprising NAB or ANAB

In one aspect, provided herein are compositions comprising NAB or ANAB.

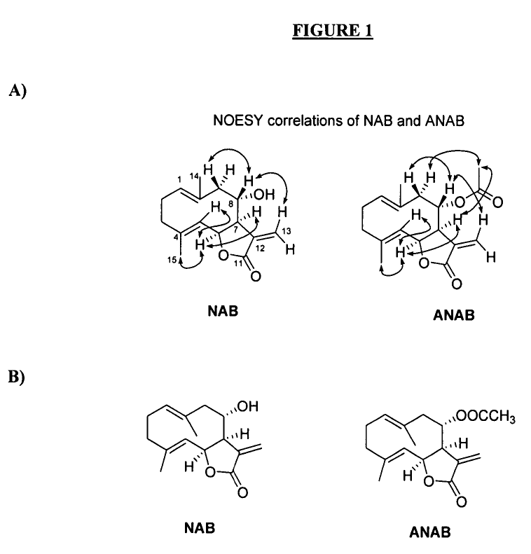

The structures of NAB and ANAB are shown in FIG. 1. NAB and ANAB can be readily obtained from certain plants. For example, NAB and/or ANAB can be isolated from other components in ethanol extracts from I. britannica flowers utilizing chromatographic techniques known to those of skill in the art. An exemplary isolation of NAB and ANAB utilizing silica gel and SEPHADEX LH-20 is described in Section 8 below.

In certain embodiments, compositions provided herein comprise an amount of NAB or ANAB effective to induce apoptosis in a cancer cell.

In some embodiments, the composition is an Inula britannica extract.

NAB and/or ANAB containing compositions can be purified, isolated or purified and isolated. In certain embodiments, the compositions are 90, 91, 92, 93, 94, 95, 96, 97, 98, 98.5, 99 or 99.5% pure. By “pure” is meant that the compositions comprise that amount of the compounds of the composition. For instance, the composition comprising NAB and/or ANAB would comprise at least would comprise at least 90, 91, 92, 93, 94, 95, 96, 97, 98, 98.5, 99 or 99.5% of either or of both compounds relative to other compounds in the composition. Purity can be assessed by any means known to those of skill in the art such as HPLC, for instance by % area under the curve.

In some embodiments, compositions provided herein are NAB and/or ANAB enriched compositions. By “enriched” is meant that the compositions comprise components of a natural source of NAB and/or ANAB, such as, for instance a plant or plant part, or product thereof such as a plant extract or plant part extract, including, for example, an I. britannica flower extract, wherein the concentration of NAB and/or concentration of ANAB relative to one or more components in the natural source or product thereof are greater than that found in the natural source or product thereof. In certain embodiments, the NAB and/or ANAB enriched compositions comprise about 10%, about 15%, about 20%, about 25%, about 30%, about 35%, about 40%, about 45%, about 50%, about 55%, about 60%, about 65%, about 70%, about 75%, about 80% or about 85% NAB and/or ANAB.

In the compositions of the invention, the amount of each component can be calculated by weight, by molar amount or by any other technique known to those of skill in the art.

In some embodiments, a composition comprising NAB or ANAB can be prepared by adding isolated NAB or ANAB to a food product or to a natural product, such as, for example, a plant extract.

In certain embodiments and depending on the manner of use, a composition of the invention can be, but is not limited to, in the form of a dietary supplement, a nutraceutical or a pharmaceutical composition.

### 7.1.1. Dietary Supplements and Nutraceuticals

In various embodiments, depending on the intended use and without limitation, a composition of the invention can be in the form of a dietary supplement or nutraceutical. Generally, a dietary supplement is consumed by a subject independent of any food composition, unlike a food additive which is incorporated into a food composition during the processing, manufacture, preparation, or delivery of the food composition, or just before its consumption. A dietary supplement provides, in addition to nutrition, a therapeutic or prophylactic function to the consumer. A “nutraceutical,” as used herein refers to a product prepared, isolated or purified from a natural source, such as a plant or plant product, not usually associated with food, for instance an Inula britannica flower, intended to be administered to a mammal to have physiological benefit or to prevent or ameliorate a condition or disorder in the mammal, that is, the nutraceutical provides a benefit other than a nutritional benefit, if any.

In various embodiments, the composition of the invention typically comprises one or more consumable fillers or carriers. The term “consumable” means the filler or carrier that is generally suitable for, or is approved by a regulatory agency of the Federal or a state government, for consumption by animals, and more particularly by humans. In certain embodiments, the meaning of the term “dietary supplement” or “nutraceutical” is the meaning of those terms as defined by a regulatory agency of the Federal or a state government, including the United States Food and Drug Administration.

As provided herein, the dietary supplement or nutraceutical can be used as an anti-cancer agent. For example, it can be used as an anti-oxidant in any condition that involves the action of free radicals. It can, for example, be used to induce apoptosis in a cancer cell or inhibit proliferation of a cancer cell. The cancer cell can, for example, be a colon cancer cell, breast cancer cell, leukemia cell, gastric cancer cell. It can, for example, be used to activate intracellular calpain and/or intracellular caspase-12 activity in a cancer cell.

Typically, a dietary supplement or nutraceutical as provided herein are intended to be orally taken or consumed. The a dietary supplement or nutraceutical can be in a solid form or a liquid form.

For example, a composition as provided herein, such as a dietary supplement or nutraceutical, can be a reconstitutable powder that, when reconstituted with a liquid, such as drinking water, can provide a beverage. In another embodiment, a composition as provided herein can be incorporated into other foodstuff, such as but not limited to cooking oil, frying oil, salad oil, margarine, mayonnaise or peanut butter. Oils containing the compounds of the invention can be emulsified and used in a variety of water-based foodstuffs, such as drinks. Accordingly, in one embodiment, compositions of the invention can be a beverage, such as but not limited to fortified mineral water, fortified distilled water, a fruit juice-based beverage, a shake, a milk-based beverage, a dairy product-based beverage, a yoghurt-based beverage, a carbonated water-based beverage, an alcoholic drink, a coffee-based beverage, a green tea-based beverage, a black tea-based beverage, a grain-based beverage, a soybean-based beverage, or a beverage based on plant extracts.

In addition to beverages, the compositions of the present invention may be combined with other foodstuff, for example, syrups, starches, grains, or grain flour.

The dietary supplement or nutraceutical compositions can further comprise any number of other beneficial components. Non-limiting examples of such optional components are essential fatty acids, vitamins and minerals. These components are well known to those of skill in the art. Additional disclosure describing the contents and production of food compositions comprising such components may be found in e.g., U.S. Pat. Nos. 5,834,048; 5,817,350; 5,792,461; 5,707,657 and 5,656,312, each of which is incorporated herein by reference in their entirety, and the like. In certain embodiments, the dietary supplement or nutraceutical compositions can further comprise, for example, vitamins, precursors, and derivatives thereof, minerals, and amino acids.

### 7.1.2. Pharmaceutical Compositions

In certain embodiments, provided herein are compositions comprising NAB or ANAB, as described above, wherein the composition is a pharmaceutical composition. Pharmaceutical compositions of the invention comprise a prophylactically or therapeutically effective amount of a composition or compound described herein, and typically one or more pharmaceutically acceptable carriers or excipients.

In this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Examples of suitable pharmaceutical carriers are described in Remington: Science and Practice of Pharmacy, 21^{st} ed., Lippincott Williams & Wilkins, Philadelphia Pa. (2005) and Ansel's Pharmaceutical Dosage Forms and Drug Delivery Systems, 8^{th} ed., Lippincott Williams & Wilkins, Philadelphia Pa. (2004).

Typical pharmaceutical compositions comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition depends on a variety of factors well known in the art including, but not limited to, the way in which the dosage form will be administered to a patient and the specific active ingredients in the dosage form. The pharmaceutical composition, if desired, can also contain minor amounts of wetting or emulsifying agents, or pH buffering agents.

This invention further encompasses anhydrous pharmaceutical compositions and dosage forms comprising active ingredients, since water can facilitate the degradation of some compounds.

A pharmaceutical composition of the invention is formulated to be compatible with its intended route of administration. Examples of routes of administration include, but are not limited to, parenteral, e.g., intravenous, intradermal, subcutaneous, oral (e.g., inhalation), intranasal, transdermal (topical), transmucosal, intra-tumoral, intra synovial and rectal administration. In a specific embodiment, the composition is formulated in accordance with routine procedures as a pharmaceutical composition adapted for intravenous, subcutaneous, intramuscular, oral, intranasal or topical administration to human beings. In a preferred embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

Generally, the ingredients of pharmaceutical compositions as provided herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. Typical dosage forms of the pharmaceutical compositions comprising NAB or ANAB, or a pharmaceutically acceptable salt, solvate or hydrate thereof lie within the range of from about 1 mg to about 1000 mg per day, given as a single once-a-day dose in the morning but preferably as divided doses throughout the day taken with food.

### 7.1.3. Unit Dosage Forms

In some embodiments, the compositions as provided herein can be in a unit dosage form. Preferably, a unit dosage form is a nutraceutical or pharmaceutical composition. Unit dosage forms of the invention comprise a prophylactically or therapeutically effective amount of NAB or ANAB and typically one or more consumable and/or physiologically or pharmaceutically acceptable carriers or excipients, as described above.

In certain other embodiments, unit dosage forms comprise an amount of NAB or ANAB, or compositions thereof, effective to induce apoptosis in a cancer cell.

In some embodiments, unit dosage forms comprise an amount of NAB or ANAB effective to inhibit proliferation of a cancer cell.

The invention further encompasses unit forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Different effective amounts may be applicable for different conditions. Unit dosage forms can, for example, take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent preferably in purified form, together with a suitable amount of carrier so as to provide the form for proper administration to the patient. The formulation should suit the mode of administration. In a preferred embodiment, the unit dosage forms are sterile and in suitable form for administration to a subject, preferably an animal subject, more preferably a mammalian subject, and most preferably a human subject.

The composition, shape, and type of dosage forms of the invention will typically vary depending on their use. For example, the prophylactically and therapeutically effective dosage form may vary among different types of cancer. Similarly, a parenteral dosage form may contain smaller amounts of NAB and/or ANAB than an oral dosage form used to treat the same disease or disorder. These and other ways in which specific dosage forms encompassed by this invention will vary from one another will be readily apparent to those skilled in the art. See, e.g., Remington: Science and Practice of Pharmacy, 21^{st} ed., Lippincott Williams & Wilkins, Philadelphia Pa. (2005); Ansel's Pharmaceutical Dosage Forms and Drug Delivery Systems, 8^{th} ed., Lippincott Williams & Wilkins, Philadelphia Pa. (2004).

In some embodiments, an article of manufacture is provided that can simplify the administration of NAB or ANAB or compositions thereof to a subject. A typical article of manufacture of the invention comprises a unit dosage form of NAB or ANAB or composition thereof. In one embodiment, the unit dosage form is a container, preferably a sterile container, containing an effective amount of NAB or ANAB or composition thereof and a pharmaceutically acceptable carrier or excipient. The article of manufacture can further comprise a label or printed instructions regarding the use of NAB or ANAB or composition thereof or other informational material that advises the dietitian, physician, technician, consumer, subject, or patient on how to appropriately prevent or treat the disease or disorder in question. In other words, the article of manufacture includes instruction means indicating or suggesting a dosing regimen including, but not limited to, actual doses, monitoring procedures, and other monitoring information. In a specific embodiment, the article of manufacture comprises a container containing an effective amount of NAB or ANAB or composition thereof and a pharmaceutically acceptable carrier or excipient. As with any pharmaceutical product, dietary supplement or nutraceutical, the packaging material and container included in the article of manufacture are designed to protect the stability of the product during storage and shipment.

Article of manufacture of the invention can further comprise devices that are useful for administering the unit dosage forms. Examples of such devices include, but are not limited to, syringes, drip bags, patches, and inhalers.

Articles of manufacture of the invention can further comprise pharmaceutically acceptable vehicles or consumable vehicles that can be used to administer the active ingredient, that is, NAB or ANAB or composition thereof. For example, if an active ingredient is provided in a solid form that must be reconstituted for parenteral or oral/enteral administration, the article of manufacture can comprise a sealed container of a suitable vehicle in which the active ingredient can be dissolved. For parenteral administration, a particulate-free sterile solution is preferred. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 7.1.4. Oral Dosage Forms

Compositions as provided herein can, for example, be suitable for oral administration, and orally consumable compositions including but not limited to dietary supplements of the invention, can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington: Science and Practice of Pharmacy, 21^{st} ed., Lippincott Williams & Wilkins, Philadelphia Pa. (2005); Ansel's Pharmaceutical Dosage Forms and Drug Delivery Systems, 8^{th} ed., Lippincott Williams & Wilkins, Philadelphia Pa. (2004).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents. Other ingredients that can be incorporated into the dietary supplement or pharmaceutical compositions of the present invention, may include, but are not limited to, vitamins, amino acids, an antioxidant, a botanical extract, metal salts, and minerals.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical/nutraceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions, dietary supplements, and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition, dietary supplement, or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions, dietary supplements, nutraceuticals and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions, dietary supplements, nutraceuticals and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions, dietary supplements, nutraceuticals or dosage forms into which they are incorporated.

In certain embodiments, NAB or ANAB in a composition as provided herein can be in a delayed release form. For example, the active ingredient can be administered by controlled release means or delivery devices that are well known to those of skill in the art, including, but not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference in its entirety.

### 7.1.5. Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. Compounds that increase the solubility of one or more of the active ingredients disclosed herein can also be incorporated into the parenteral dosage forms of the invention.

### 7.1.6. Transdermal, Topical & Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington: Science and Practice of Pharmacy, 21^{st} ed., Lippincott Williams & Wilkins, Philadelphia Pa. (2005); Ansel's Pharmaceutical Dosage Forms and Drug Delivery Systems, 8^{th} ed., Lippincott Williams & Wilkins, Philadelphia Pa. (2004). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington: Science and Practice of Pharmacy, 21^{st} ed., Lippincott Williams & Wilkins, Philadelphia Pa. (2005) and Ansel's Pharmaceutical Dosage Forms and Drug Delivery Systems, 8^{th} ed., Lippincott Williams & Wilkins, Philadelphia Pa. (2004).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

### 7.2. Methods Using NAB, ANAB and Compositions Thereof

In one aspect, provided herein are methods of using NAB, ANAB or composition thereof as anti-proliferation agents. As demonstrated in the Examples below, NAB and ANAB are each demonstrated to have antiproliferative effects, including, for example, inducing apoptosis in cancer cells. Adverse health conditions, diseases and disorders which can be prevented, treated, managed, or ameliorated by administering an effective amount of one or more compounds or compositions of the invention include, but are not limited to, proliferative disorders and symptoms thereof.

### 7.2.1. Proliferative Disorders

NAB, ANAB or composition thereof can be used to prevent, treat, manage, or ameliorate a proliferative disorder or one or more symptoms thereof. In certain embodiments, provided herein are methods for preventing, treating, managing, or ameliorating one or more symptoms of a non-cancerous disorder associated with cellular hyperproliferation, particularly of epithelial cells (e.g., as in asthma, COPD, pulmonary fibrosis, bronchial hyperresponsiveness, psoriasis, lymphoproliferative disorder, and seborrheic dermatitis), and endothelial cells (e.g., as in restenosis, hyperproliferative vascular disease, Behcet's Syndrome, atherosclerosis, and macular degeneration), said methods comprising administering to a subject in need thereof NAB, ANAB or composition thereof.

In a specific embodiment, the invention provides methods for preventing, managing, treating, or ameliorating a non-cancerous disorder associated with cellular hyperproliferation (e.g., Behcet's Syndrome, sarcoidosis, keloids, pulmonary fibrosis, and renal fibrosis) or one or more symptoms thereof, said methods comprising of administering to a subject in need thereof a prophylactically or therapeutically effective amount of NAB, ANAB or composition thereof.

The present invention provides methods for preventing, treating, managing, or ameliorating cancer or one or more symptoms thereof, said methods comprising administering NAB, ANAB or composition thereof to a subject in need thereof.

In a specific embodiment, the invention provides a method of preventing, treating, managing, or ameliorating cancer or one or more symptoms thereof, said method comprising administering to a subject in need thereof a dose of a prophylactically or therapeutically effective amount of NAB, ANAB or composition thereof.

The compounds of the invention can be used in vitro or ex vivo for the management, treatment or amelioration of certain cancers, including, but not limited to leukemias and lymphomas, such treatment involving, for example, autologous stem cell transplants. This can involve a multi-step process in which the subject's autologous hematopoietic stem cells are harvested and purged of all cancer cells, the patient's remaining bone-marrow cell population is then eradicated via the administration of a high dose of a compound of the invention with or without accompanying high dose radiation therapy, and the stem cell graft is infused back into the subject. Supportive care is then provided while bone marrow function is restored and the subject recovers.

In further embodiments, cancers that can be prevented, managed, treated or ameliorated in accordance with the methods of the invention include, but are not limited to, neoplasms, tumors (malignant and benign) and metastases, or any disease or disorder characterized by uncontrolled cell growth. The cancer may be a primary or metastatic cancer. Specific examples of cancers that can be prevented, managed, treated or ameliorated in accordance with the methods of the invention include, but are not limited to, cancer of the head, neck, eye, mouth, throat, esophagus, chest, bone, lung, colon, rectum, stomach, prostate, breast, ovaries, kidney, liver, pancreas, and brain. Additional cancers include, but are not limited to, the following: leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease, and inflammatory breast cancer; gastric or stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma. For a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America, each of which is incorporated herein by reference in its entirety for all purposes.

In certain embodiments, the methods provided comprise contacting a cancer cell with an amount of NAB, ANAB or composition thereof effective to induce apoptosis in the cancer cell. In some embodiments, the activated apoptosis is a calcium-mediated apoptosis.

In certain embodiments, the methods provided comprise contacting a cancer cell with NAB, ANAB or composition thereof in an amount effective to activate calpain and/or caspase-12.

In certain embodiments, methods are provided for the inhibition cancer proliferation and/or inducement of apoptosis in a cancer cell, the method comprising contacting the cancer cell with 6β-O-(2-methylbutyryl)-britannilactone or neobritannilactone A.

### 7.2.2. Dosage & Frequency of Administration

The amount of NAB, ANAB or composition thereof which will be effective in the prevention, treatment, management, relief, or amelioration of an cancer or one or more symptoms thereof will vary with the nature and severity of the cancer and the route by which the active ingredient is administered. The frequency and dosage will also vary according to factors specific for each subject or patient depending on the specific therapy (e.g., therapeutic or prophylactic agents) administered, the severity of the cancer, the route of administration, as well as age, body, weight, response, and the past medical history of the patient. Effective doses may be extrapolated from dose-response curves derived from in vitro or animal model test systems. Suitable regiments can be selected by one skilled in the art by considering such factors and by following, for example, dosages reported in the literature and recommended in the Physician's Desk Reference (57th ed., 2003).

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

In general, the recommended daily dose range of NAB or ANAB for the conditions described herein lie within the range of from about 0.01 mg of NAB or ANAB to about 1000 mg NAB or ANAB per day. These amounts can, for example, be given as a single once-a-day dose or as divided doses throughout a day. In one embodiment, the daily dose is administered twice daily in equally divided doses. Specifically, a daily dose range should be from about 5 mg to about 500 mg per day, more specifically, between about 10 mg and about 200 mg per day. In managing the subject or patient, the therapy should be initiated at a lower dose, perhaps about 1 mg to about 25 mg, and increased if necessary up to about 200 mg to about 1000 mg per day as either a single dose or divided doses, depending on the subject or patient's global response. It may be necessary to use dosages of the active ingredient outside the ranges disclosed herein in some cases, as will be apparent to those of ordinary skill in the art. Furthermore, it is noted that the dietitian, clinician or treating physician will know how and when to interrupt, adjust, or terminate therapy in conjunction with individual patient responses and conditions, as will be readily known by those of ordinary skill in the art. Similarly, amounts sufficient to prevent, manage, treat or ameliorate such disorders, but insufficient to cause, or sufficient to reduce, adverse effects associated with the compounds of the invention are also encompassed by the above described dosage amounts and dose frequency schedules. Further, when a subject or patient is administered multiple dosages of a compound of the invention, not all of the dosages need be the same. For example, the dosage administered to the subject or patient may be increased to improve the prophylactic or therapeutic effect of the compound or it may be decreased to reduce one or more side effects that a particular subject or patient is experiencing.

In a specific embodiment, the dosage of NAB or ANAB or composition thereof administered to prevent, treat, manage, or ameliorate a cancer or one or more symptoms thereof in a patient is about 5 μg/kg, about 50 μg/kg, about 100 μg/kg, about 150 μg/kg, preferably about 250 μg/kg, about 500 μg/kg, about 1 mg/kg, about 5 mg/kg, about 10 mg/kg, about 25 mg/kg, about 50 mg/kg, about 75 mg/kg, about 100 mg/kg, about 125 mg/kg, about 150 mg/kg, or about 200 mg/kg or more of a patient's body weight. In another embodiment, the dosage of NAB or ANAB or composition thereof administered to prevent, treat, manage, or ameliorate a cancer or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

### 7.3. Biological Assays

Several aspects of NAB or ANAB or composition thereof f can be tested in vitro, in a cell culture system, and in an animal model organism, such as a rodent animal model system, for the desired therapeutic activity prior to use in humans. For example, assays which can be used to determine whether administration of a specific composition is indicated, include cell culture assays in which a patient tissue sample is grown in culture, and exposed to or otherwise contacted with a composition, and the effect of such composition upon the tissue sample is observed. The tissue sample can be obtained by biopsy from the patient. This test allows the identification of the therapeutically most effective therapy (e.g., prophylactic or therapeutic agent(s)) for each individual patient. In various specific embodiments, in vitro assays can be carried out with representative cells of cell types involved in a disorder (e.g., cancer cells), to determine if a composition of the invention has a desired effect upon such cell types. As an alternative to the use of tissue, tissue samples, cancer cell lines can be used in in vitro assays. Examples of cancer cell lines that can be utilized in in vitro assays include, but are not limited to, the MCF-7 breast cancer cell line, the MCF-7/ADR multi-drug resistant breast cancer cell line, the HT114 human melanoma cell line, the MES/DOX doxorubicenresistant human uterine sarcoma cell line, the HT29 human colorectal cell line, the HCT-116 human colorectal cell line, the A549 human lung Carcinoma cell line and the BXPC-3 human pancreas primary adenocarcinoma cell line, including cell lines described in the Examples below.

NAB, ANAB or composition thereof can be assayed for their ability to induce the expression and/or activation of a gene product (e.g., cellular protein or RNA) and/or to induce signal transduction in cancer cells. The induction of the expression or activation of a gene product or the induction of signal transduction pathways in cancer cells (in particular tubulin-binding agent resistant cancer cells) can be assayed by techniques known to those of skill in the art including, e.g., ELISAs, flow cytometry, Northern blot analysis, Western blot analysis, RT-PCR kinase assays and electrophoretic mobility shift assays. NAB or ANAB or composition thereof can also be assayed for their ability to modulate immune cell proliferation, endothelial and cell cancer cell proliferation. Techniques known to those in art, including, but not limited to, ³H-thymidine incorporation, trypan blue cell counts, and fluorescence activated cell sorting (“FACS”) analysis. NAB or ANAB or composition thereof can also be assayed for their ability to induce cytolysis. Cytolysis can be assessed by techniques known to those in art, including, but not limited to, ⁵¹Cr-release assays.

NAB or ANAB or composition thereof can be tested in suitable animal model systems prior to use in humans. Such animal model systems include, but are not limited to, rats, mice, chicken, cows, monkeys, pigs, dogs, rabbits, etc. Any animal system well-known in the art may be used. In a specific embodiment of the invention, NAB or ANAB or composition thereof is tested in a mouse model system. Such model systems are widely used and well-known to the skilled artisan. Pharmaceutical compositions of the invention can be administered repeatedly. Several aspects of the procedure may vary including, but not limited to, temporal regime for administration of NAB or ANAB or composition thereof.

The anti-cancer activity of NAB or ANAB or composition thereof can be determined using any suitable animal model, including, but not limited to, SCID mice with a tumor or injected with malignant cells. Examples of animal models for lung cancer include, but are not limited to, lung cancer animal models described by Zhang & Roth (1994, In Vivo 8(5):755-69) and a transgenic mouse model with disrupted p53 function (see, e.g., Morris et al., 1998, J La State Med Soc 150(4):179-85). An example of an animal model for breast cancer includes, but is not limited to, a transgenic mouse that overexpresses cyclin D1 (see, e.g., Hosokawa et al., 2001, Transgenic Res 10(5):471-8). An example of an animal model for colon cancer includes, but is not limited to, a TCR b and p53 double knockout mouse (see, e.g., Kado et al., 2001, Cancer Res 61(6):2395-8). Examples of animal models for colorectal carcinomas include, but are not limited to, Apc mouse models (see, e.g., Fodde & Smits, 2001, Trends Mol Med 7(8):369-73 and Kuraguchi et al., 2000, Oncogene 19(50):5755-63).

Further, any assays known to those skilled in the art can be used to evaluate the prophylactic and/or therapeutic utility of NAB or ANAB or composition thereof for the disorders disclosed herein.

The toxicity and/or efficacy of NAB or ANAB or composition thereof can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage of the compositions and compounds of the invention for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography (HPLC) and radioimmunasssay (RIA). The pharmacokinetics of a prophylactic or therapeutic can be determined, e.g., by measuring parameters such as peak plasma level (Cₘₐₓ), area under the curve (AUC, which is measured by plotting plasma concentration of the agent versus time, and reflects bioavailability), half-life of the compound (t_{1/2}), and time at maximum concentration.

Efficacy in preventing or treating a proliferative disorder such as cancer may be demonstrated, e.g., by detecting the ability of the compositions and compounds of the invention to reduce one or more symptoms of the proliferative disorder, to reduce the proliferation of cancerous cells, to reduce the spread of cancerous cells, or to reduce the size of a tumor. Efficacy in preventing or treating an inflammatory disorder may be demonstrated, e.g., by detecting the ability of the compositions and compounds of the invention to reduce one or more symptoms of the inflammatory disorder, to decrease T cell activation, to decrease T cell proliferation, to modulate one or more cytokine profiles, to reduce cytokine production, to reduce inflammation of a joint, organ or tissue or to improve quality of life. Changes in inflammatory disease activity may be assessed through tender and swollen joint counts, patient and physician global scores for pain and disease activity, and the ESR/CRP. Progression of structural joint damage may be assessed by quantitative scoring of X-rays of hands, wrists, and feet (Sharp method). Changes in functional status in humans with inflammatory disorders may be evaluated using the Health Assessment Questionnaire (HAQ), and quality of life changes are assessed with the SF-36.

### 8. EXAMPLES

The following examples are intended only to further illustrate the invention and are not intended to limit the scope of the invention as defined by the claims.

### 8.1. Example 1

This example presents an exemplary isolation of neobritannilactone B (NAB) and/or acety neobritannilactone B (ANAB) from I. britannica extracts.

Silica gel (130-270 mesh), SEPHADEX LH-20 and RP-18 (60 μm) (Sigma Chemical Co., St. Louis, Mo.) were used for column chromatography. All solvents used were purchased from Fisher Scientific (Springfield, N.J.). ¹H and ¹³C NMR spectra were recorded on a U-400 instrument (Varian Inc., Palo Alto, Calif.). Chemical shifts are expressed in parts per million (δ) using TMS as internal standard. CD₃OD and CDCl₃ were purchased from Aldrich Chemical Co. (Allentown, Pa.). HRFAB-MS was run on a JEOL HX-110 double focusing mass spectrometer. FT-IR was performed on a Perkin-Elmer spectrum BX system. UV was on a Cary 300 Bio UV-Visible spectrophotometer. Optical rotations were determined in MeOH solutions on a Perkin-Elmer 141 Polarimeter.

Plant Material. The dried flowers of I. britannica were purchased from Shanghai Drugs Company, cultivated in the Jiansu Province of China. It was identified by Professor Zhi Wei Wang, College of Pharmacy, Fudan University, Shanghai, China. Voucher specimens were deposited in the Laboratory of Phytochemistry, College of Pharmacy, Fudan University, Shanghai, China.

Extraction and Isolation. The flowers (10 kg) of I. britannica were extracted three times with 95% EtOH at room temperature. The EtOAc soluble part of the EtOH extract was chromatographed on a silica gel column, packed in CHCl₃ using a CHCl₃-MeOH gradient solvent system. The fractions from CHCl₃-MeOH (20:1 to 10:1) were evaporated under vacuum and repeatedly chromatographed on silica gel and SEPHADEX LH-20 columns, to give seven sesquiterpene lactones including neobritannilactone B (NAB) (102 mg) and acetyl neobritannilactone B (ANAB) (12 mg). The five other sesquiterpene lactones isolated were 6β-O-(2-methylbutyryl)-britannilactone (63 mg); neobritannilactone A (15 mg); britannilactone (21 mg); 1-O-acetylbritannilactone (1.1 g); and 1,6-O,O-diacetylbritannilactone (32 mg). The structures of britannilactone, 1-O-acetylbritannilactone and 1,6-O,O-diacetylbritannilactone were identified by their known physical and spectroscopic data. Characterization of the other four compounds were as follows.

Neobritannilactone B (4-hydroxy-3α,4,5,8,9,11α-hexahydro-6,10-dimethyl-3-methylene-cyclodeca[b]furan-2(3H)-one; NAB): amorphous powder; [α]²⁰_{D}-15.2 (c 0.06, MeOH); UV (MeOH) λₘₐₓ (log ε) 215.0 (3.92); IR (LF) υₘₐₓ 3430, 3100, 1730, 1660, 820 cm⁻¹; ¹H and ¹³C NMR data, see Table I; DI EI HRMS m/z 248.1418 [M+H]⁺ (calcd for C₁₅H₂₁O₃ 248.1413).

Acetyl neobritannilactone B (ANAB): amorphous powder; [α]²⁰_{D} -17.3 (c 0.08, MeOH); UV (MeOH) λₘₐₓ (log ε) 217.0 (4.32); ¹H and ¹³C NMR data, see Table I; DI EI HRMS m/z 290.1516 [M+H]⁺ (calcd for C₁₇H₂₃O₄ 291.1518).

The structure of NAB was formulated as C₁₅H₂₀O₃ by HREIMS (obsd. 248.1418, calcd. 248.1413, [M+H]⁺). The presence of the α-methylene γ-lactone group was evidenced from the ¹H NMR signals at δ_{H} 5.59 and 6.39 ppm and ¹³C NMR signals at δ_{C} 120.4, 138.3, and 170.2 ppm. Further analysis of its ¹H and ¹³C NMR data (Table I) as well as the observed correlation in the ¹H-¹H COSY, TOCSY, HMQC, and HMBC spectra suggested that the structure of NAB would be closely related to that of ANAB. Comparison of the ¹H and ¹³C NMR data of NAB and ANAB revealed that the difference between these two isolates is the absence of an acetyl group in NAB. This is consistent with its determined molecular formula. The relative configuration of NAB and ANAB were established by interpretation of both 1D-NOE difference and 2D-NOESY NMR spectroscopic data. FIG. 1.

Structures of 6β-O-(2-methylbutyryl)-britannilactone and neobritannilactone A were determined using similar techniques including mass spectrometry IR, ¹H and ¹³C NMR, aided by ¹H-¹H COSY, TOCSY, HMQC, HMBC and DEPT spectra, as appropriate. See also Bai et al. (2005) “Sesquiterpene Lactones from Inula britannica and Their Cytotoxic and Apoptotic Effects on Human Cancer Cell Lines.” J. Nat. Prod. (in press), incorporated by reference herein in its entirety for all purposes.

6β-O-(2-methylbutyryl)-britannilactone: amorphous powder; [α]²⁰_{D} +46.0 (c 0.18, MeOH); UV (MeOH) λₘₐₓ (log ε) 212.0 (4.11); IR (LF) υₘₐₓ 3382, 1762, 1749, 1654 cm⁻¹; HRFABMS m/z 351.2169 [M+H]⁺ (calcd for C₂₀H₃₁0₅ 351.2171).

Neobritannilactone A: amorphous powder; [α]²⁰_{D} +14.0 (c 0.08, MeOH); IR (LF) υₘₐₓ 3450, 1755, 1729 cm⁻¹; HRFABMS m/z 311.1859 [M+H]⁺ (calcd for C₁₇H₂₇O₅ 311.1858).

TABLE I
¹H, ¹³C NMR and HMBC Data for NAB85 and ANAB (CD₃OD)^{a}

| | NAB | | | | | ANAB | | | | |
|---|---|---|---|---|---|---|---|---|---|---|
| No. | δ_{H} | | δ_{C} | | HMBC (H to C) | δ_{H} | | δ_{C} | | HMBC (H to C) |
| 1 | 4.83 | m | 129.2 | d | | 4.83 | d (11.6) | 130.6 | d | |
| 2 | 2.27 | m | 26.1 | t | 1, 3, 10, 14 | 2.23 | m | 26.1 | t | 1, 3, 10, 14 |
| | 2.35 | m | | | | 2.27 | m | | | |
| 3 | 1.95 | m | 39.4 | t | 2, 4, 5, 15 | 2.04 | m | 39.3 | t | 2, 4, 5, 15 |
| | 2.39 | m | | | | 2.34 | m | | | |
| 4 | | | 142.6 | s | | | | 142.4 | s | |
| 5 | 4.78 | d (10.2) | 127.5 | d | 3, 7, 15 | 4.71 | d (10.0) | 127.2 | d | 3, 7, 15 |
| 6 | 5.23 | t (8.6) | 75.1 | d | 4 | 5.06 | t (10.0) | 75.5 | d | 4, 7 |
| 7 | 2.78 | m | 53.6 | d | | 2.85 | m | 52.4 | d | |
| 8 | 4.61 | m | 71.7 | d | 6, 10 | 5.66 | m | 71.5 | d | 6, 10, 16 |
| 9 | 2.05 | d (13.2) | 47.8 | t | | 2.31 | m | 43.8 | t | 1, 7, 8, 10 |
| | 2.76 | d (13.2) | | | | 2.76 | dd (4.8, 14.4) | | | |
| 10 | | | 135.8 | s | | | | 134.2 | s | |
| 11 | | | 138.3 | s | | | | 136.7 | s | |
| 12 | | | 170.2 | s | | | | 169.8 | s | |
| 13 | 5.59 | d (3.6) | 120.4 | t | 7, 12 | 5.54 | d (2.4) | 120.9 | t | 7, 12 |
| | 6.39 | d (3.6) | | | 7, 11, 12 | 6.25 | d (3.6) | | | 7, 11, 12 |
| 14 | 1.63 | s | 19.5 | q | 1, 9, 10 | 1.44 | s | 18.8 | q | 1, 9, 10 |
| 15 | 1.74 | d (1.6) | 17.4 | q | 3, 4, 5 | 1.70 | d (1.6) | 17.4 | q | 3, 4, 5 |
| 16 | | | | | | | | 169.6 | s | |
| 17 | | | | | | 2.01 | s | 20.9 | q | 16 |

^{a}Carbon multiplicities were determined by DEPT experiments (s = C, d = CH, t = CH₂, q = CH₃); Figures in parentheses denote J values (Hz).

### 8.2. Example 2

This example demonstrates that NAB and ANAB are effective for inhibiting human cancer cell proliferation and inducing apoptosis in cancer cells.

Each of the seven sesquiterpene lactones isolated in Example 1, above, were tested for anti-proliferative and apoptosis-inducing activities, and are referred to in this section by a numerical designation as follows: 6β-O-(2-methylbutyryl)-britannilactone (1); neobritannilactone A (2); NAB (3); ANAB (4); britannilactone (5); 1-O-acetylbritannilactone (6); and 1,6-O,O-diacetylbritannilactone (7).

Cell Culture and Chemicals. The COLO 205 and HT 29 cell lines were isolated from human colon adenocarcinoma (ATCC CCL-222 and HTB-38); human promyelocytic leukemia (HL-60) cells were obtained from American Type Culture Collection (Rockville, Md.). The human AGS gastric carcinoma cell lines (CCRC 60102) were obtained from the Food Industry Research and Development Institute (Hsinchu, Taiwan). Cell lines were grown at 37° C. in 5% CO₂ dioxide atmosphere in RPMI for COLO 205, HT-29, and HL-60 cells and DMEM/F12 for AGS cells, all supplemented with 10% heat-inactivated fetal bovine serum (GIBCO BRL, Grand Island, N.Y.) (100 units/mL of penicillin, 100 μg/mL of streptomycin), and 2 mM L-glutamine (GIBCO BRL). Selected compounds were dissolved in dimethyl sulfoxide (DMSO). Propidium iodide was obtained from Sigma Chemical Co. (St. Louis, Mo.).

Determination of Cell Viability. Human cancer cells were treated either with DMSO (0.01%) or the selected compounds (5-100 μM). Cell viability was determined at 24 h based on trypan blue exclusion assay. The viability percentage was calculated based on the percentage of unstained cells. Suspensions of cells were diluted 1:1 with 0.5% trypan blue solution. Stained and unstained cells were counted in a hemocytometer.

Flow Cytometry. Human cancer cells (2×10⁵) were cultured in 60-mm Petri dishes and incubated for 24 h. The apoptotic cells (sub-G1) in the selected compounds and treated cells were measured by flow cytometry analysis. Then cells were harvested, washed with PBS, resuspended in 200 μL of PBS, and fixed in 800 μL of iced 100% ethanol at −20° C. After being left to stand overnight, the cell pellets were collected by centrifugation, resuspended in 1 mL of hypotonic buffer (0.5% Triton X-100 in PBS and 0.5 μg/mL RNase), and incubated at 37° C. for 30 min. Then 1 mL of propidium iodide solution (50 μg/mL) was added and the mixture was allowed to stand on ice for 30 min. Fluorescence emitted from the propidium iodide-DNA complex was quantitated after excitation of the fluorescent dye by FACScan cytometry (Becton Dickinson, San Jose, Calif.).

Results. Human cancer cells were treated with different concentrations (5-100 μM) of selected compounds for 24 h and viability of the cells was determined by typical blue exclusion. As shown in Table II, significant cytotoxicity was observed in all types of human cancer cells treated with NAB (3) and ANAB (4). NAB and ANAB have significant influence on the viability of COLO 205, HT-29, HL-60, and AGS cells, with observed IC₅₀ values of 14.3, 56.1, 27.4, and 21.4 μM, respectively, for NAB and 14.7, 57.0, 16.2, 5.4 μM, respectively. NAB and ANAB strongly inhibited HL-60 and AGS cells.

TABLE II
Effects of NAB and ANAB on the growth of human cancer cells^{a}

| | IC₅₀ (μM) | | | |
|---|---|---|---|---|
| Compound | COLO 205 | HT-29 | HL-60 | AGS |
| 1 | 58.7 ± 1.32 | 48.1 ± 2.26 | 47.1 ± 1.55 | 31.3 ± 1.8 |
| 2 | 97.9 ± 1.16 | >100 | 85.8 ± 4.97 | >100 |
| 3 | 14.3 ± 0.84 | 56.1 ± 5.16 | 27.4 ± 4.41 | 21.4 ± 1.76 |
| 4 | 14.7 ± 1.18 | 57.0 ± 2.53 | 16.2 ± 2.93 | 5.4 ± 0.41 |
| 5 | >100 | >100 | >100 | >100 |
| 6 | >100 | 96.0 ± 12.52 | >100 | >100 |
| 7 | 56.7 ± 3.66 | 76.6 ± 5.50 | 35.3 ± 0.44 | 66.4 ± 6.29 |

^{a}Human cancer cells were treated with various concentrations of compounds (1-7) for 24 h. The numbers of viable cells were determined by counting the trypan blue-excluding cells in a hemocytometer. Three samples were analyzed in each group, and values represent the mean ± SE.

The compounds isolated from Inula were tested for their effects on the apoptotic ratio in human cancer cells. A sub-G1 (sub-2N) DNA peak, which has been suggested to be apoptotic DNA (Telford et al. (1992) Cytometry 13, 137-142), was detected in cells that were treated with selected compounds (1-7), washed, and stained with propidium iodide.

As shown in Table III, compounds NAB (3) and ANAB (4) appear to be potent apoptosis-inducing agents for COLO 205, HT-29, AGS, and HL-60 cells. These apoptotic effects were found to be dose-dependent. The percentages of apoptotic COLO 205, HT-29, HL-60, and AGS cells were 41.62 and 76.87%; 66.54 and 69.70%; 77.54 and 95.17%; 11.78 and 9.89% after 24 h of incubation with NAB and ANAB(25 μM), respectively. ANAB appears to be more potent and induced dose-dependent cell apoptosis in all types of human cancer cells.

TABLE III
Induction of apoptosis in human cancer cells by compounds 1-7^{a}

| | Concentration | Human Cancer Cells (apoptotic ratio) % | | | |
|---|---|---|---|---|---|
| Compound | (μM) | COLO 205 | HT-29 | HL-60 | AGS |
| Control | — | 4.38 ± 1.29 | 3.92 ± 0.47 | 7.62 ± 2.76 | 9.91 ± .11 |
| 1 | 5 | 5.33 ± 0.78 | 4.24 ± 0.40 | 6.41 ± 0.79 | 10.89 ± 1.69 |
| 1 | 10 | 5.86 ± 0.32 | 3.68 ± 0.47 | 5.36 ± 0.61 | 11.38 ± 1.12 |
| 1 | 25 | 15.20 ± 1.02 | 3.93 ± 0.16 | 10.53 ± 3.34 | 17.76 ± 0.76 |
| 1 | 50 | 27.21 ± 5.51 | 5.16 ± 0.93 | 28.60 ± 2.67 | 28.48 ± 1.20 |
| 1 | 100 | 36.24 ± 0.76 | 6.40 ± 0.28 | 27.05 ± 0.54 | 27.10 ± 0.54 |
| 2 | 5 | 4.08 ± 0.21 | 3.94 ± 1.05 | 6.68 ± 1.51 | 9.23 ± 0.22 |
| 2 | 10 | 6.51 ± 2.28 | 4.24 ± 0.24 | 6.31 ± 0.98 | 9.83 ± 2.27 |
| 2 | 25 | 4.28 ± .93 | 5.17 ± 1.70 | 6.19 ± 0.01 | 10.57 ± 2.11 |
| 2 | 50 | 5.44 ± 0.47 | 4.09 ± 0.77 | 6.63 ± 2.21 | 9.41 ± 2.83 |
| 2 | 100 | 25.50 ± 3.46 | 7.73 ± 3.22 | 16.31 ± 1.63 | 9.81 ± 2.30 |
| 3 | 5 | 10.73 ± 2.31 | 13.58 ± 0.87 | 9.82 ± 1.90 | 9.55 ± 3.05 |
| 3 | 10 | 14.12 ± 1.06 | 24.46 ± .88 | 21.93 ± 2.81 | 8.66 ± 0.80 |
| 3 | 25 | 41.62 ± 6.34 | 66.54 ± 1.58 | 77.57 ± 7.06 | 11.78 ± 1.40 |
| 3 | 50 | 89.11 ± 1.47 | 82.27 ± 1.68 | 96.94 ± 0.57 | 15.95 ± 1.12 |
| 3 | 100 | 96.81 ± 0.51 | 87.02 ± 0.30 | 98.29 ± 0.15 | 19.22 ± 0.57 |
| 4 | 5 | 15.47 ± 1.69 | 20.38 ± .17 | 22.93 ± 1.34 | 6.16 ± 1.17 |
| 4 | 10 | 34.72 ± 2.72 | 47.56 ± 0.00 | 39.71 ± 2.85 | 7.87 ± 3.03 |
| 4 | 25 | 76.87 ± 2.39 | 69.70 ± .22 | 95.17 ± 1.61 | 9.89 ± 4.44 |
| 4 | 50 | 96.92 ± 0.20 | 81.43 ± 1.46 | 97.74 ± 0.44 | 13.64 ± 0.28 |
| 4 | 100 | 98.66 ± 0.10 | 82.47 ± 2.06 | 98.05 ± .30 | 43.11 ± 0.99 |
| 5 | 5 | 11.90 ± 3.61 | 5.75 ± 1.77 | 7.22 ± 1.54 | 7.93 ± 1.71 |
| 5 | 10 | 12.28 ± 5.24 | 6.16 ± 1.23 | 5.64 ± 2.00 | 10.37 ± 0.98 |
| 5 | 25 | 12.79 ± 1.84 | 6.49 ± 1.07 | 5.24 ± 0.67 | 10.16 ± .28 |
| 5 | 50 | 15.54 ± 2.14 | 6.59 ± 0.54 | 8.01 ± 1.34 | 8.97 ± 0.66 |
| 5 | 100 | 28.31 ± 1.89 | 7.55 ± .08 | 12.83 ± .06 | 10.37 ± 0.66 |
| 6 | 5 | 13.44 ± .25 | 5.36 ± 1.80 | 4.05 ± 0.31 | 15.37 ± 6.22 |
| 6 | 10 | 11.32 ± .421 | 6.81 ± 2.14 | 7.26 ± 2.15 | 17.28 ± 2.29 |
| 6 | 25 | 16.48 ± 3.75 | 11.68 ± 3.02 | 4.37 ± 0.57 | 14.66 ± 1.38 |
| 6 | 50 | 16.59 ± 0.71 | 9.26 ± 1.80 | 5.69 ± 0.60 | 14.84 ± .58 |
| 6 | 100 | 35.79 ± 1.36 | 14.87 ± .90 | 22.19 ± 4.14 | 18.85 ± .68 |
| 7 | 5 | 6.94 ± .33 | 11.19 ± 3.98 | 6.14 ± .20 | 7.49 ± .88 |
| 7 | 10 | 8.69 ± 1.65 | 11.15 ± 2.84 | 8.67 ± 2.06 | 5.79 ± 0.64 |
| 7 | 25 | 26.95 ± 0.30 | 11.50 ± .92 | 21.13 ± 0.37 | 11.29 ± 2.16 |
| 7 | 50 | 15.44 ± .08 | 13.08 ± 0.63 | 27.02 ± 2.69 | 10.79 ± 2.79 |
| 7 | 100 | 17.82 ± 2.26 | 20.88 ± .09 | 22.24 ± 1.06 | 8.12 ± .26 |

^{a}Cells were harvested 24 h after treatment, and apoptosis was quantified by flow cytometry. The method of flow cytometry used is described under Materials and Method. Three samples were analyzed in each group and the results were presented as means ±SE.

Taken together, these data demonstrate that NAB and ANAB exhibit strong cytotoxicity and apoptotic-inducing activity on cancer cells.

### 8.3. Example 3

This example characterizes the apoptosis-inducing effects of NAB and ANAB in human gastric carcinoma cells.

### 8.3.1. Procedures

Cell Survival Assay. The human AGS gastric carcinoma cell lines (CCRC 60102) were maintained as described in Section 8.2 above. For assays, cells (5×10⁴) were plated in 35-mm Petri dishes. The next day, the medium was changed and NAB and ANAB were added. Control cells were treated with DMSO to a final concentration of 0.05% (v/v). At the end of incubation, cells were harvested for cell count using a hemocytometer.

Flow Cytometry. AGS cells (2×10⁵) were cultured in 60-mm Petri dishes and incubated for 24 h. The cells were then harvested, washed with PBS, resuspended in 200 μL of PBS, and fixed in 800 μL of iced 100% ethanol at −20° C. After being left to stand overnight, the cell pellets were collected by centrifugation, resuspended in 1 mL of hypotonic buffer (0.5% Triton X-100 in PBS and 0.5 μg/mL RNase), and incubated at 37° C. for 30 min. Next, 1 mL of propidium iodide solution (50 μg/mL) was added, and the mixture was allowed to stand on ice for 30 min. Fluorescence emitted from the propidium iodide-DNA complex was quantitated after excitation of the fluorescent dye by FACScan cytometry (Becton Dickinson, San Jose, Calif.).

DNA Extraction and Electrophoresis Analysis. The AGS human cancer cells were harvested, washed with phosphate-buffered saline (PBS), and then lysed with digestion buffer containing 0.5% sarkosyl, 0.5 mg/mL proteinase K, 50 mM tris(hydroxy methyl)aminomethan (pH 8.0), and 10 mM EDTA at 56° C. overnight and treated with RNase A (0.5 μg/mL) for 3 h at 56° C. The DNA was extracted by phenol/chloroform/isoamyl (25:24:1) before loading and was analyzed by 2% agarose gel electrophoresis. The agarose gels were run at 50 V for 120 min in Tris-borate/EDTA electrophoresis buffer (TBE). Approximately 20 μg of DNA was loaded in each well and visualized under UV light, and photographed.

Western Blotting. The nuclear and cytosolic proteins were isolated from AGS cells after treatment with 60 μM for 0, 3, 6, 9, 12, 18, and 24 h. The total proteins were extracted via the addition of 200 μL of gold lysis buffer (50 mM Tris-HCl, pH 7.4; 1 mM NaF; 150 mM NaCl; 1 mM EGTA; 1 mM phenylmethanesulfonyl fluoride; 1% NP-40; and 10 μg/mL leupeptin) to the cell pellets on ice for 30 min, followed by centrifugation at 10,000 g for 30 min at 4° C. The cytosolic fraction (supernatant) proteins were measured by Bio-Rad protein assay (Bio-Rad Laboratories, Munich, Germany). The samples (50 μg of protein) were mixed with 5× sample buffer containing 0.3 M Tris-HCl (pH 6.8), 25% 2-mercaptoethanol, 12% sodium dodecyl sulfate (SDS), 25 mM EDTA, 20% glycerol, and 0.1% bromophenol blue. The mixtures were boiled at 100° C. for 5 min and were subjected to 12% SDS-polyacrylamide minigels at a constant current of 20 mA. Subsequently, electrophoresis was ordinarily carried out on SDS-polyacrylamide gels. For electrophoresis, proteins on the gel were electrotransferred onto an immobile membrane (PVDF; Millipore Corp., Bedford, Mass.) with transfer buffer composed of 25 mM Tris-HCl (pH 8.9), 192 mM glycine, and 20% methanol. The membranes were blocked with blocking solution containing 20 mM Tris-HCl and then immunoblotted with primary antibodies including, anti-Bcl-X_{L}, anti-β-actin (Transduction Laboratory, Lexington, Ky.), anti-PARP (UBI, Inc., Lake Placid, N.Y.), at room temperature for 1 h. Detection was achieved by measuring the chemiluminescence of blotting agent (ECL, Amersham Corp., Arlington Heights, Ill.), after exposure of the filters to KODAK X-OMAT films.

### 8.3.2. Results

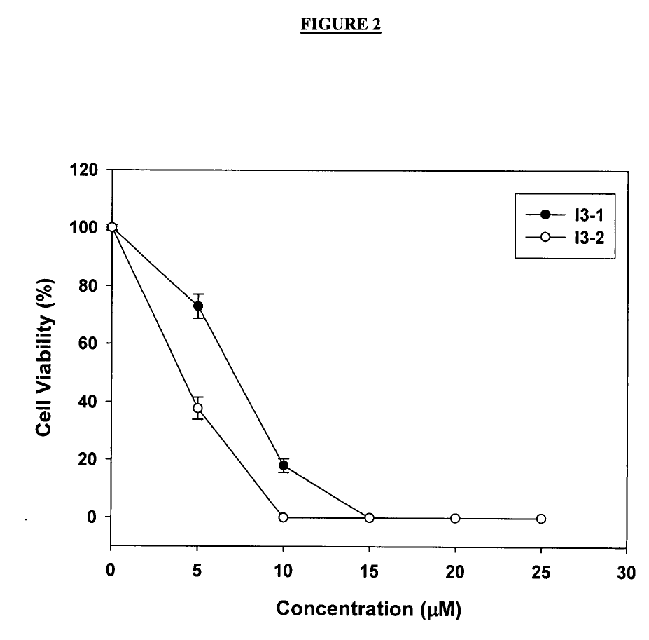

To test the effects of NAB and ANAB on cell viability, human gastric carcinoma cells were treated with different concentrations of NAB and ANAB. After 24 h treatment, the number of live cells was determined by trypan blue exclusion assay. FIG. 2. Both compounds were potent inhibitors of cell viability. ANAB was found to have an IC₅₀ of 4.2 μM and NAB was found to have an IC₅₀ of 7.6 [μM. These results indicate that NAB and ANAB inhibit AGS cell growth.

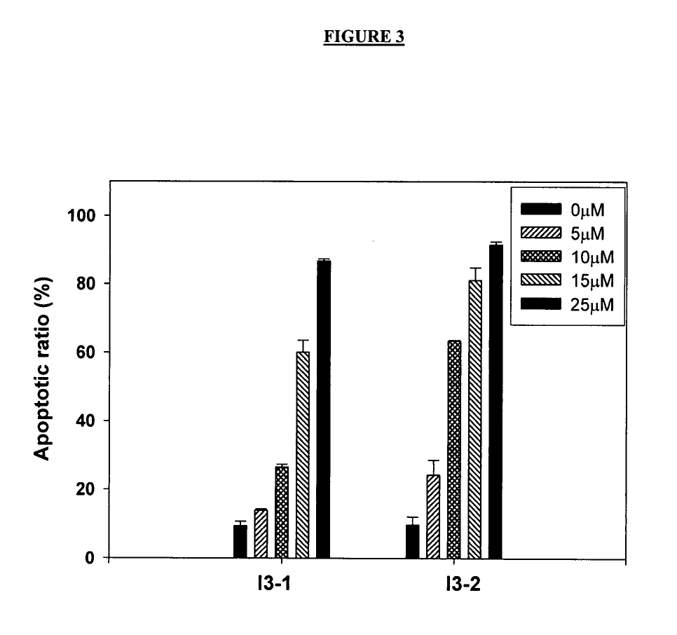

The cytotoxic effects of NAB and ANAB in AGS cells were analyzed to assess their consistency with the specific changes that characterize apoptosis, such as DNA fragmentation. To investigate the induction of a sub-G1 cell population, the DNA content of AGS cells treated with various concentrations of NAB and ANAB was analyzed by flow cytometry. Cells were treated with NAB and ANAB, and stained with propidum iodide. As seen in FIG. 3, the percentages of apoptotic AGS cells was 9, 14, 27, 63, and 86% after incubation with 0, 5, 10, 15, and 25 μM NAB for 24 h, respectively and 9, 24, 64, 81, and 92% after incubation with 0, 5, 10, 15, and 25 μM ANAB for 24 h, respectively.

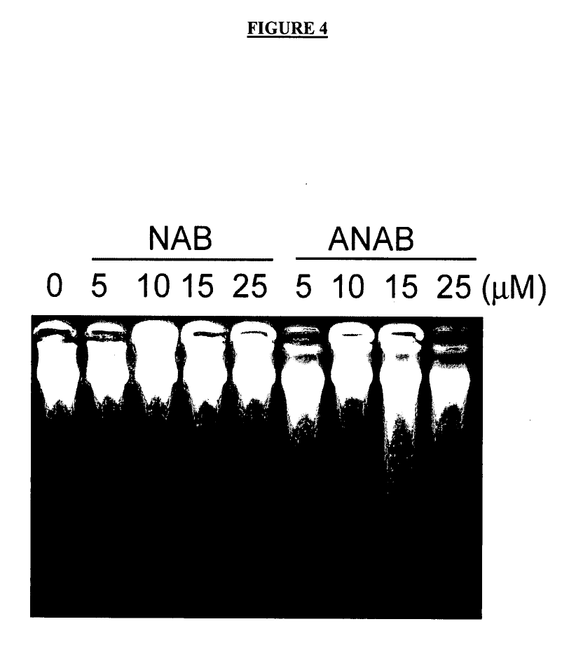

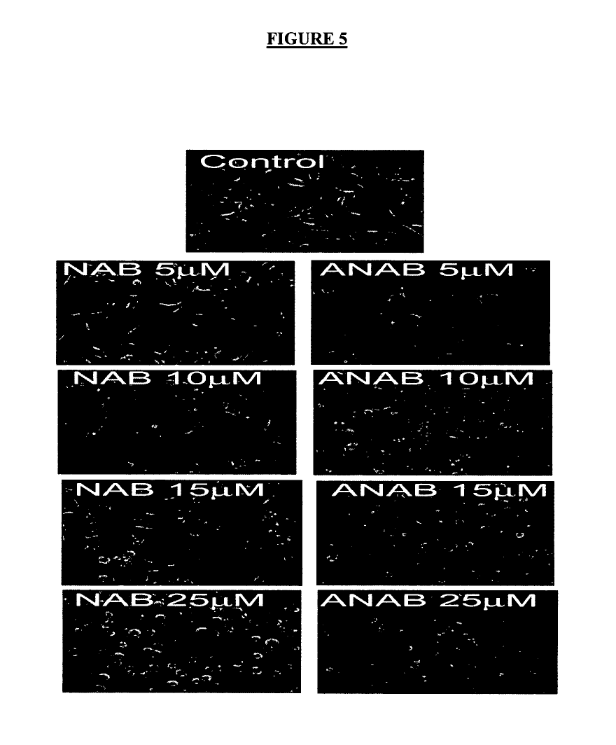

Physiological cell death is characterized by apoptotic morphology, including chromatin condensation, membrane blebbing, internucleosome degradation of DNA, and apoptotic body formation. Nucleosomal DNA ladders visible on agarose gel after staining with ethidium bromide are typical of apoptotic cell samples. To investigate whether the cytotoxic effects of NAB and ANAB observed in AGS cells were due to the presence of apoptotic cell death, cells were treated with hexamethyl-Dewar-benzene (HMDB) (0-25 μM) for 24 h and DNA fragmentation analyses were performed. As shown in FIG. 4, significant DNA ladders were observed in AGS cells after 15 μM of ANAB treatment for 24 h. Within 24 h of treatment with 10 μM ANAB, cells clearly exhibited significant morphological changes and chromosomal condensation, which is indicative of apoptotic cell death. FIG. 5. These results indicate that the cytotoxic action of NAB and ANAB is due to the induction of apoptosis.

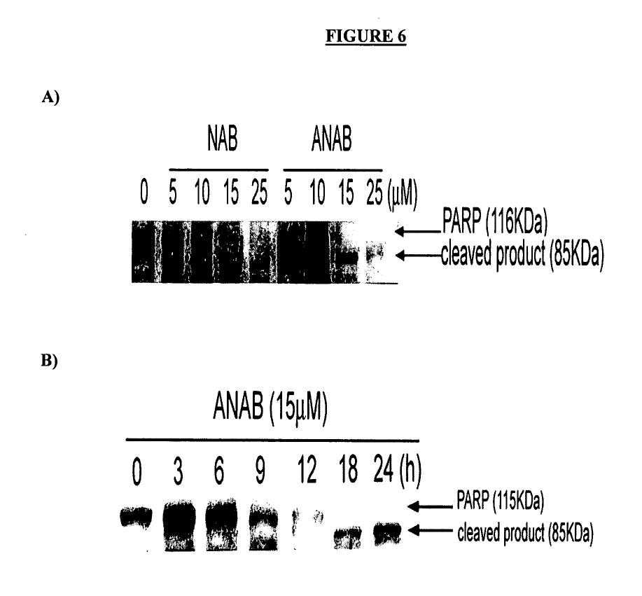

Activation of caspase-3 leads to the cleavage of a number of proteins, one of which is poly-(ADP-ribose) polymerase (PARP). A hallmark of apoptosis is that PARP (116 kDa) is cleaved to produce an 85-kDa fragment. PARP cleavage was examined in cells treated with NAB or ANAB for 24. As shown in FIG. 6, the PARP proteolytic fragment was found in cells treated either with NAB or with ANAB (A). Treatment of AGS cells with 15 μM ANAB for more than 24 h caused a time-dependent proteolytic cleavage of PARP, with accumulation of the 85-kDa fragment and concomitant disappearance of the full-size 116-kDa protein.

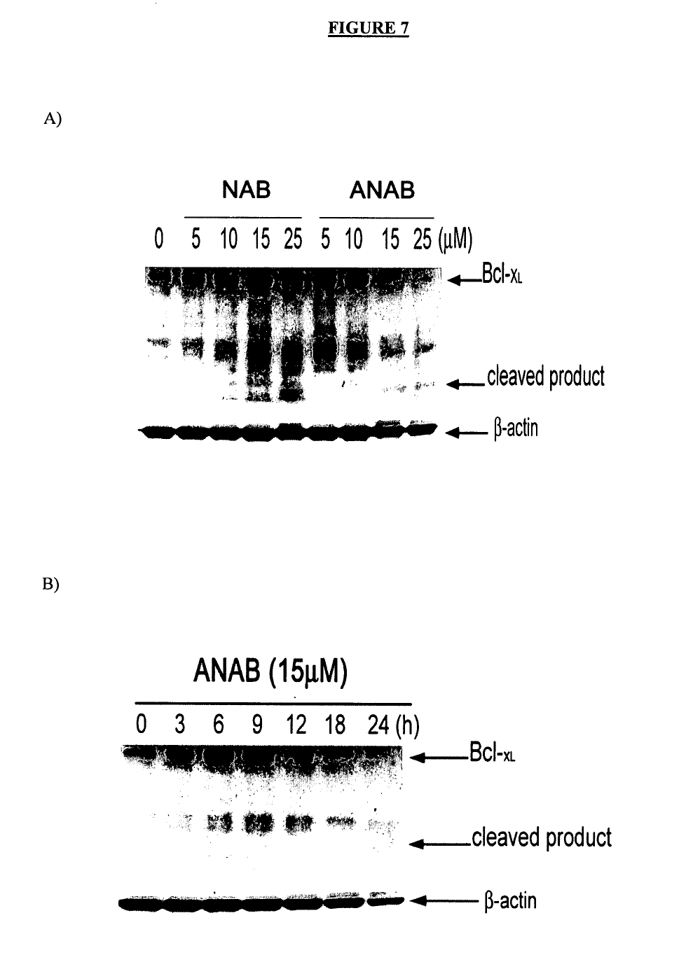

Several gene products are known to be important in controlling the apoptotic process. The imbalance of expression of anti- and pro-apoptotic proteins after a stimulus is one of the major mechanisms underlying the ultimate fate of cells in the apoptotic process. Bcl-X_{L} expression in tumor cells, for instance, confers resistance against chemotherapeutic drugs. The expression of anti-apoptotic protein Bcl-X_{L} was therefore examined in cancer cells treated with various concentrations of NAB or ANAB for 24 hours. A dose-dependent decrease of Bcl-X_{L} and increase of the cleaved product of Bcl-X_{L} were observed in NAB- and ANAB-treated cells. FIG. 7A. A marked and significant change in the expression of Bcl-X_{L} observed at 12 h in ANAB-treated AGS cells. FIG. 7B.

Taken together, these results indicate that NAB and ANAB induce apoptosis in cancer cells.

All publications, patents and patent applications mentioned in this specification are herein incorporated by reference into the specification to the same extent as if each individual publication, patent or patent application was specifically and individually indicated to be incorporated herein by reference. Citation or discussion of a reference herein shall not be construed as an admission that such is prior art to the present invention.

While the invention has been described in terms of various preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed:

1. A method of inducing apoptosis in a cancer cell comprising contacting the cancer cell with an amount of isolated neobritannilactone B (NAB) or isolated acetyl neobritannilactone B (ANAB) sufficient to induce apoptosis.

2. The method of claim 1, wherein the cancer cell is a colon cancer cell, a leukemia cell or a gastric cancer cell.

3. The method of claim 1, wherein the cancer cell is contacted with NAB.

4. The method of claim 1, wherein the cancer cell is contacted with ANAB.

5. A method for inhibiting the proliferation of a cancer cell in a mammal in need thereof, comprising administering to the mammal an amount of NAB or ANAB effective to inhibit proliferation of the cancer cell.

6. The method of claim 5, wherein the amount of NAB or ANAB administered per kilogram of body mass is between about 5 μg/kg to about 10 mg/kg

7. A method of preventing or treating a cancer in a mammal in need thereof, comprising administering to the mammal an amount of a NAB or ANAB effective to prevent or treat the cancer.

8. The method of claim 7, wherein the amount of NAB or ANAB administered per kilogram of body mass is between about 5 μg/kg to about 10 mg/kg

9. The method of claim 7, wherein the cancer cell is a colon cancer cell, a leukemia cell or a gastric cancer cell.

10. The method of claim 7, wherein NAB is administered to the mammal.

11. The method of claim 7 wherein ANAB is administered to the mammal.

12. A pharmaceutical composition comprising NAB and a pharmaceutically acceptable carrier or excipient.

13. A pharmaceutical composition comprising ANAB and a pharmaceutically acceptable carrier or excipient.
